(19) United States
(12) Patent Application Publication
(10) Pub. No.: US 2022/0265409 A1
(43) Pub. Date: Aug. 25, 2022

(54) DENTAL DEVICES FOR SIMULTANEOUS INTRA-ORAL AND EXTRA-ORAL TREATMENTS

(71) Applicants: Dustin Lee Cohen, Los Angeles, CA (US); Stacey Cohen, Los Angeles, CA (US)

(72) Inventors: Dustin Lee Cohen, Los Angeles, CA (US); Stacey Cohen, Los Angeles, CA (US)

(21) Appl. No.: 17/677,377

(22) Filed: Feb. 22, 2022

Related U.S. Application Data
(60) Provisional application No. 63/152,194, filed on Feb. 22, 2021.

(51) Int. Cl. A61C 19/06; A61N 5/06

(52) U.S. Cl. CPC A61C 19/066; A61N 5/0603; A61N 5/0616; A61N 2005/0606; A61N 2005/0659; A61N 2005/0663; A61N 2005/0632; A61N 2005/0647

(57) ABSTRACT

A dental device for simultaneous intra-oral and extra oral treatment can include an intra- oral treatment structure configured to treat one or more intra-oral tissues with at least one intra- oral structure treatment. The device can also include an extra-oral treatment structure attached to and extending distally from the intra-oral treatment structure such that at least a portion of the extra-oral treatment structure is configured to be extra-oral in use, wherein the extra-oral treatment structure is configured to treat one or more extra oral structures with at least one extra- oral structure treatment.

### CROSS-REFERENCE TO RELATED APPLICATIONS

This application claims priority to and the benefit of U.S. Provisional Application No. 63/152,194, filed Feb. 22, 2021, the entire contents of which are herein incorporated by reference in their entirety.

### FIELD

This disclosure relates to dental devices.

### BACKGROUND

Soft tissue, e.g., gums and lip tissue for example, can be treated with red light therapy methods, whereas teeth can be bleached using a blue light therapy combined with bleaching solution. However, these treatments are separate, time consuming, and costly.

Such conventional methods and systems have generally been considered satisfactory for their intended purpose. However, there is still a need in the art for improved dental devices. The present disclosure provides a solution for this need.

### SUMMARY

A dental device for simultaneous intra-oral and extra oral treatment can include an intra- oral treatment structure configured to treat one or more intra-oral tissues with at least one intra- oral structure treatment. The device can also include an extra-oral treatment structure attached to and extending distally from the intra-oral treatment structure such that at least a portion of the extra-oral treatment structure is configured to be extra-oral in use, wherein the extra-oral treatment structure is configured to treat one or more extra oral structures with at least one extra- oral structure treatment.

The intra-oral treatment structure can include a tooth bleaching tray configured to receive teeth of a patient. The tooth bleaching tray can include one or more first treatment lights. The tooth bleaching tray can include a gum extension configured to overlay at least a portion of a gum. The gum extension can include one or more second treatment lights. Any other suitable intra-oral structures are contemplated herein.

The one or more first treatment lights can include one or more blue lights. The one or more second treatment lights can include one or more red or infrared lights. Any suitable number or position of lights, colors, and/or types are contemplated herein.

The extra-oral treatment structure can include a lip covering configured to receive and/or cover one or both lips of a patient. The lip covering can include one or more one or more lip treatment lights. The lip treatment lights can include one or more red lights and/or infrared lights. In certain embodiments, a vent can be defined through the extra-oral treatment structure and/or the intra-oral treatment structure and configured to allow a patient to breathe through the vent when the device is in use. The device can include a battery disposed in a battery structure extending distally from the extra-oral treatment structure. The vent may pass through the battery structure in certain embodiments.

In accordance with at least one aspect of this disclosure, a method can include simultaneously applying intra-oral treatment while applying extra-oral treatment to the lips of a patient. Applying intra-oral treatment can include bleaching teeth using a first light treatment, and applying extra-oral treatment can include light treating lips using a second light treatment different from the first light treatment. The method can include using a single dental device to apply both treatments simultaneously.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is a top plan view of an embodiment of a device in accordance with this disclosure;

FIG. 2 is a side elevation view of the embodiment of FIG. 1; and

FIG. 3 is a front elevation view of the embodiment of FIG. 1.

### DETAILED DESCRIPTION

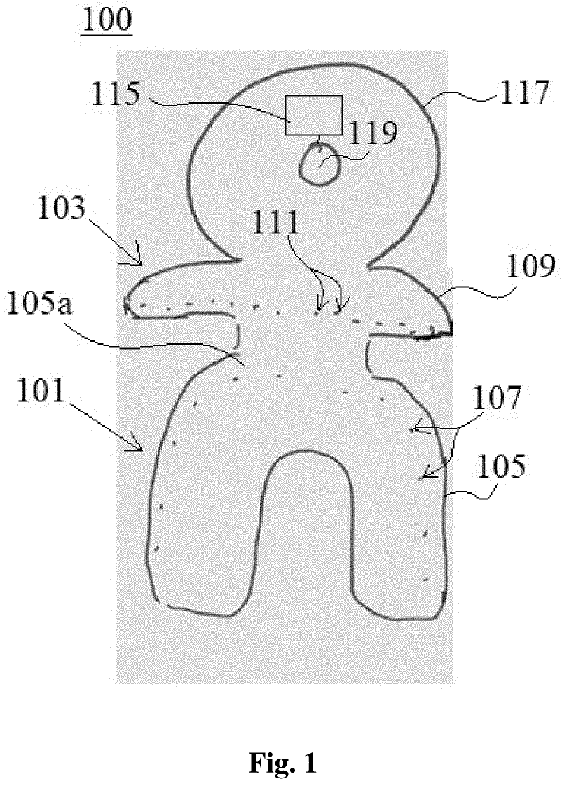

Reference will now be made to the drawings wherein like reference numerals identify similar structural features or aspects of the subject disclosure. For purposes of explanation and illustration, and not limitation, an illustrative view of an embodiment of a device in accordance with the disclosure is shown in FIG. 1 and is designated generally by reference character 100.

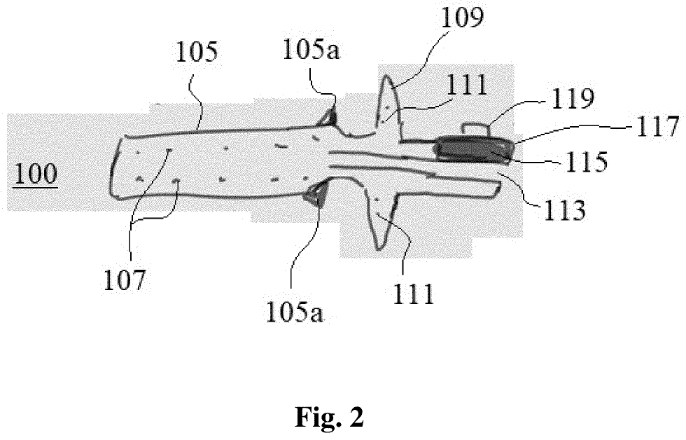

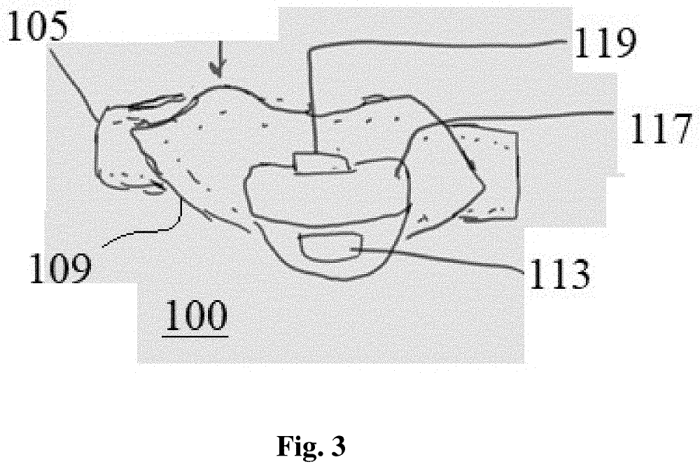

Other embodiments and/or aspects of this disclosure are shown in FIGS. 2 and 3. Certain embodiments described herein can be used to provide efficient and simultaneous treatment of intra-oral and extra-oral tissues, for example.

Referring to FIGS. 1-3, a dental device 100 for simultaneous intra-oral and extra oral treatment can include an intra-oral treatment structure 101 configured to treat one or more intra- oral tissues (e.g., teeth, gums) with at least one intra-oral structure treatment (e.g., light treatment). The device 100 can also include an extra-oral treatment structure 103 attached to and extending distally from the intra-oral treatment structure 101 such that at least a portion of the extra-oral treatment structure 103 is configured to be extra-oral in use. The extra-oral treatment structure 103 can be configured to treat one or more extra oral structures (e.g., lips) with at least one extra-oral structure treatment (e.g., light treatment).

The intra-oral treatment structure 101 can include a tooth bleaching tray 105 configured to receive teeth of a patient. The tooth bleaching tray 105 can include one or more first treatment lights 107. The tooth bleaching tray 105 can include a gum extension 105a configured to overlay at least a portion of a gum. The gum extension 105a can include one or more second treatment lights. Any other suitable intra-oral structures are contemplated herein.

The one or more first treatment lights 107 can include one or more blue lights. The one or more second treatment lights (e.g., associated with gum extensions 105a) can include one or more red or infrared lights. Any suitable number or position of lights, colors, and/or types are contemplated herein for either or both of the first treatment lights and/or second treatment lights. For example, in certain embodiments, the first treatment lights can be any suitable tooth treatment light and the second treatment lights can be any suitable soft tissue and/or gum treatment light.

The extra-oral treatment structure 103 can include a lip covering 109 configured to receive and/or cover one or both lips of a patient. The lip covering 109 can include one or more one or more lip treatment lights 111. The lip treatment lights 111 can include one or more red lights and/or infrared lights, for example. Any other suitable treatment lights are contemplated herein.

In certain embodiments, a vent 113 can be defined through the extra-oral treatment structure 103 and/or the intra-oral treatment structure 101 and configured to allow a patient to breathe through the vent 113 when the device 100 is in use. The device 100 can include a battery 115 disposed in a battery structure 117 extending distally from the extra-oral treatment structure 103. The vent 113 may pass through the battery structure 117 in certain embodiments, e.g., as shown. The lights, e.g., lights 107, 111 can be operatively connected to the battery 115 via a button 119 and/or any other suitable switch such that power can be selectively applied to the lights.

In accordance with at least one aspect of this disclosure, a method can include simultaneously applying intra-oral treatment while applying extra-oral treatment to the lips of a patient. Applying intra-oral treatment can include bleaching teeth using a first light treatment, and applying extra-oral treatment can include light treating lips using a second light treatment different from the first light treatment. The method can include using a single dental device to apply both treatments simultaneously.

Embodiments can include a red light lip/gum therapy and blue light tooth bleaching device. Embodiments can use red light therapy for soft tissue benefits and blue light therapy to enhance tooth bleaching into a single device. Embodiments allow for a decreased overall treatment time and an increased cosmetic benefit compared to separate devices.

Embodiments can have both intra-oral and extra-oral components including an intra-oral tray (e.g., made of silicone, rubber, or plastic or any other suitable material) lined with both red and blue (or just blue) LED lights where bleaching gel is placed and teeth bite into the tray, for example. The extra-oral portion can cover the lips and be lined with red and infrared light LEDs, for example. This can be used with or without a gel made to go on the lips and can also include the battery/power source with one or more on/off and/or mode buttons (e.g., for controlling different groups of lights), for example. An optional vent can extend from the intra-oral portion to the extra-oral portion for ease of breathing. Red light (e.g., 620-750 nm range) and Infrared Light (e.g., 750-810 nm range) can help to promote increased circulation, along with collagen and elastin production. This can be effective in increasing the volume of the lips, aid in repair of dry and cracked lips, minimizing wrinkles in the lips, and providing more pleasant looking lips overall. The red and infrared light can also have benefits for the gingiva, including decreased inflammation, decreased redness, & overall more pleasant looking gums.

The Blue light (e.g., 380-495 nm range) can help to increase the effectiveness of tooth bleaching products along with a reduction in bacteria. The effect of the blue light can help to provide fresher breath and a more cosmetically pleasing smile full of brighter teeth.

An embodiment of a process is described. One can place carbamide peroxide gel or hydrogen peroxide tooth bleaching gel in tray, apply optional topical gel/ointment to lips, turn on device, and place in the mouth so that teeth are biting into the intra-oral tray and the outer portion of the device covers the lips. Treatment time can last between about 2-15 minutes, or any other suitable time. The device can be configured to shut off automatically. One can then take the device out, clean with soap and water, rinse the mouth, and repeat daily.

Causes of tooth discoloration include external factors and internal factors. For example, smoking and darkly tinted food/drink can leave surface stain on teeth (e.g., from coffee, tea, beets, blueberries). Internal factors can include medications, trauma, genetics, or blood particles. Causes of lip breakdown can include sun damage, dry weather, aging, and trauma for example.

Causes of gingiva breakdown can include bacterial infections, poor oral hygiene, and trauma for example. Embodiments may be useful to aid in treatment and/or prevention of these issues, for example.

Embodiments can be configured to allow use certain materials in conjunction with the device including bleaching gel (e.g., carbamide peroxide or hydrogen peroxide), tartar removal gel, antioxidant gels for gingiva treatment, and petroleum products or antioxidant gels for lips.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. A dental device for simultaneous intra-oral and extra oral treatment, comprising:
an intra-oral treatment structure configured to treat one or more intra-oral tissues with at least one intra-oral structure treatment; and
an extra-oral treatment structure attached to and extending distally from the intra-oral treatment structure such that at least a portion of the extra-oral treatment structure is configured to be extra-oral in use, wherein the extra-oral treatment structure is configured to treat one or more extra oral structures with at least one extra-oral structure treatment.

2. The dental device of claim 1, wherein the intra-oral treatment structure includes a tooth bleaching tray configured to receive teeth of a patient.

3. The dental device of claim 2, tooth bleaching tray includes one or more first treatment lights.

4. The dental device of claim 3, wherein the tooth bleaching tray includes a gum extension configured to overlay at least a portion of a gum.

5. The dental device of claim 4, wherein the gum extension includes one or more second treatment lights.

6. The dental device of claim 5, wherein the one or more first treatment lights include one or more blue lights, wherein the one or more second treatment lights include one or more red or infrared lights.

7. The dental device of claim 1, wherein the extra-oral treatment structure includes a lip covering configured to receive and/or cover one or both lips of a patient.

8. The dental device of claim 7, wherein the lip covering includes one or more one or more lip treatment lights.

9. The dental device of claim 8, wherein the lip treatment lights include one or more red lights and/or infrared lights.

10. The dental device of claim 1, further comprising a vent defined through the extra-oral treatment structure and/or the intra-oral treatment structure and configured to allow a patient to breathe through the vent when the device is in use.

11. The dental device of claim 10, further comprising a battery disposed in a battery structure extending distally from the extra-oral treatment structure.

12. A method, comprising:
simultaneously applying intra-oral treatment while applying extra-oral treatment to the lips of a patient.

13. The method of claim 12, wherein applying intra-oral treatment includes bleaching teeth using a first light treatment, and wherein applying extra-oral treatment includes light treating lips using a second light treatment different from the first light treatment.

14. The method of claim 13, wherein the method includes using a single dental device to apply both treatments simultaneously.
